(19) United States
(12) Patent Application Publication
(10) Pub. No.: US 2007/0059340 A1
(43) Pub. Date: Mar. 15, 2007

(54) Omega-3 Fatty Acids Encapsulated In Zein Coatings and Food Products Incorporating the Same

(76) Inventors: Anthony Bello, Battle Creek, MI (US); Norbert Gimmler, Portage, MI (US); Pradip K. Roy, Grand Rapids, MI (US)

Correspondence Address: Randall L. Shoemaker;Dickinson Wright PLLC, Ste. 2000, 38525 Woodward Ave., Bloomfield Hills, MI, 48304-2970, US

(21) Appl. No.: 11/530,633

(22) Filed: Sep. 11, 2006

Related U.S. Application Data
(60) Provisional application No. 60/715,920, filed on Sep. 9, 2005.

(51) Int. Cl. A61K 47/00; A61K 31/22

(52) U.S. Cl. 424/439; 514/547; 426/660

(57) ABSTRACT

Disclosed are processes for stabilizing omega-3 fatty acids for use in food products. The processes permit creation of a variety of food forms and food ingredients that contain omega-3 fatty acids like docosahexaenoic acid and eicosapentaenoic acid wherein these foods and food forms are stable for months without developing fishy aromas or tastes. This stability enables the incorporation of omega-3 fatty acids into food forms such as ready to eat cereals, trail mixes, chips, granola bars, toaster pastries, baked goods, cookies, crackers, fruit pieces and fruit leathers. The processes utilize a zein coating to protect and stabilize the omega-3 fatty acids.

### RELATED APPLICATIONS

This application claims the benefit of U.S. Provisional application No. 60/715,920, filed Sep. 9, 2005.

### STATEMENT REGARDING FEDERALLY SPONSORED RESEARCH

NONE

### TECHNICAL FIELD

This invention relates generally to a food having high levels of long chain polyunsaturated omega-3 fatty acids and, more particularly, to a food or food ingredient having high levels of long chain polyunsaturated omega-3 fatty acids with reduced fishy odor and improved stability.

### BACKGROUND OF THE INVENTION

Consumption of long chain polyunsaturated fatty acids is beneficial to human health. In particular, long chain polyunsaturated omega-3 fatty acids are beneficial. The three long chain polyunsaturated omega-3 fatty acids of primary interest are linolenic acid (18:3w-3), eicosapentaenoic acid (EPA) (20:5w-3), and docosahexaenoic acid (DHA) (22:6w-3). The health benefits associated with enhanced consumption of these omega-3 fatty acids include a lowering of serum cholesterol, reduction of blood pressure, reduction of the risk of heart disease, and a reduction of the risk of stroke. These omega-3 fatty acids are also essential to normal neuronal development and their depletion has been associated with neurodegenerative diseases such as Alzheimer's disease. In the human eye and retina the ratio of DHA:EPA is 5:1 and their presence is essential for normal eye development. The fatty acid DHA is also believed to be essential for optimal cognitive development in infants. Food fortified with DHA is often called “brain food” in Asian countries. Preliminary studies demonstrate that long chain polyunsaturated omega-3 fatty acids can play a role in mediating chronic inflammatory assaults and their use by individuals with mild asthma is documented to reduce the severity of the histamine response.

There are several main sources of these beneficial long chain polyunsaturated omega-3 fatty acids. Certain plants provide an abundant source of linolenic fatty acid. Marine animals, such as fish and crustaceans, and marine plants, such as micro algae, are the main sources of DHA and EPA. In particular, fatty fish such as mackerel and salmon contain high levels of DHA and EPA. Marine micro algae contain predominantly DHA. Marine micro algae have an advantage over marine animals as a source of DHA because large volumes can be produced rapidly and there is no need for the extensive acreage associated with fish farms nor is there the difficulty of ocean fishing. The omega-3 fatty acids from these sources are generally found in the form of triglycerides, i.e. one of more of the fatty acids connected to the glycerol backbone is an omega-3 fatty acid, and not in the form of free fatty acids. Both forms have the health benefits and the problems of oxidative instability. Therefore, in this specification and the associated claims no distinction will be made between these two forms of omega-3 fatty acids. The term omega-3 fatty acid refers to both the free fatty acid form and the triglyceride form unless specifically noted otherwise.

The beneficial health effects of the omega-3 fatty acids, especially EPA and DHA, require consumption of relatively large amounts of the omega-3 fatty acids making it impractical to obtain the recommended daily amount by consuming marine animals alone. Thus, both EPA and DHA have been packaged together in caplet form. Consumers do not enjoy consuming the caplets because they are large, hard to swallow and the caplets often develop an unpleasant fishy aroma and taste during storage. These unpleasant aromas and tastes arise from oxidative damage of the EPA or DHA in the caplets. Prior attempts to add the omega-3 fatty acids DHA and/or EPA directly to food products have been unsuccessful because the unstable omega-3 fatty acids rapidly give rise to a fishy taste and aroma in the food product and make it unpalatable. Typically, this fishy taste and aroma occurs either right as the food product is made or within several days to several weeks. It is believed that DHA and EPA are particularly unstable in the presence of water, oxygen and high heat, this further complicates their use in a wide variety of food products since exposure to water, oxygen and heat is difficult to avoid in their manufacture. Unlike other fatty acids these omega-3 fatty acids can not be stabilized in foods merely by adding the typical antioxidants to the foods. The unpleasant aromas and tastes are so potent that oxidative damage of DHA or EPA during a manufacturing process leading to the aromas can require a plant shut down and extensive cleaning of the equipment to remove the aroma and to prevent it from contaminating other foods. Obviously, this can be very costly and problematic for development of foods containing these beneficial omega-3 fatty acids.

It is desirable to develop a method to allow incorporation of the omega-3 fatty acids into foods that does not involve complicated processing steps or the use of unique ingredients and that promotes the shelf life of the food product. Shelf life is defined as the length of time the food product containing the omega-3 fatty acid can be stored without the development of fishy aromas or tastes. It is also desirable to develop a method that can easily be incorporated into standard food processing methods.

### SUMMARY OF THE INVENTION

In general terms, this invention provides a method for stabilizing omega-3 fatty acids so that they can be used in food products without developing fishy aromas and tastes upon storage. In one embodiment the present invention is a method for stabilizing an omega-3 fatty acid comprising the steps of: providing an edible carrier material and plating at least one omega-3 fatty acid unto the carrier material; providing a zein coating solution; applying the zein coating solution to the carrier material plated with at least one omega-3 fatty acid; and drying the zein coated carrier material. In another embodiment the present invention is a method for creating an agglomerate containing a stabilized omega-3 fatty acid comprising the steps of: providing a pre-gelatinized flour; mixing the pre-gelatinized flour with at least one omega-3 fatty acid and adding it to a agglomerating system; adding water to the mixture of pre-gelatinized flour and at least one omega-3 fatty acid to form a plurality of agglomerates; providing a zein coating solution and applying the zein coating solution to the plurality of agglomerates; and drying the plurality of zein coated agglomerates. In another embodiment the present invention is a method for stabilizing a food product comprising an omega-3 fatty acid comprising the steps of: providing a food product; applying at least one omega-3 fatty acid onto the food product; and providing a zein coating solution and applying the zein coating solution to the food product having the omega-3 fatty acid thereon. In another embodiment the present invention is a stabilized omega-3 fatty acid comprising an edible carrier material, the edible carrier material further comprising at least one omega-3 fatty acid and a zein coating, the zein coating encapsulating said carrier material and the omega-3 fatty acid. In another embodiment the present invention is a food product comprising at least one omega-3 fatty acid, the food product encapsulated in a zein coating. In another embodiment the present invention is a food product comprising an edible carrier material comprising at least one omega-3 fatty acid, said edible carrier material encapsulated in a zein coating.

These and other features and advantages of this invention will become more apparent to those skilled in the art from the detailed description of a preferred embodiment. The drawings that accompany the detailed description are described below.

### DETAILED DESCRIPTION OF A PREFERRED EMBODIMENT

As discussed above marine animals and marine plants are the main sources of EPA and DHA fatty acids. The use of marine oils from marine animals as a source of EPA and DHA is well known. Recently, a number of manufacturers have developed processes for growing marine micro algae with high efficiency. These micro algae are a great source of marine oil containing EPA and DHA at very high yields in a completely renewable process. Such micro algae derived EPA and DHA are available from a number of sources. One source of micro algae derived EPA and DHA is Martek Biosciences Corporation, Columbia, Md., USA. A second source of micro algae derived EPA and DHA is Nutrinova Nutrition Specialties and Food Ingredients, DE. The omega-3 fatty acids are available in the form of oil solutions or as free flowing powders. The oils contain anywhere from 1 to 50% by weight of marine oil the rest comprising a carrier oil. To create a free flowing powder the marine oil is typically plated out onto edible carrier material such as corn syrup solids, mannitol, calcium carbonate, whey protein isolate, gluten, pre-gelatinized starches, cellulose fiber, carbohydrates, gelatin, flour, grain, bran, or mixtures of these carrier materials. Preferably the edible carrier material has a particle size of from about 100 to about 400 microns. The plated powders are then spray dried to form a free flowing powder containing omega-3 fatty acids. One such powder is designated as HM by Martek Biosciences Corp. The omega-3 fatty acids are available as either the free fatty acids or in the triglyceride form. Generally, the triglyceride form is more stable and in the present specification and claims there will be no distinction made between whether the fatty acids are in the free form or as part of a triglyceride. All of the omega-3 fatty acids used in the examples described below, however, were in the triglyceride form.

In the present invention the inventors have discovered a method for encapsulating the marine oils in a prolamine coat which dramatically enhances the shelf life and flavor of omega-3 fatty acid containing food products. Prolamine describes a super family of cereal seed storage proteins. Prolamine is found in many cereal grains including maize, sorghum, millets, wheats, ryes, and other cereal grains. These cereal seed storage proteins are called prolamines because they tend to have high levels of the amino acids proline and glutamine. The prolamines are characterized by insolubility in water or anhydrous alcohol, but solubility in alcohol water mixtures. Some research has suggested that the group of prolamines found in maize, sorghum and millets form a specific subfamily. The maize prolamines are know in the art as zeins and they exist as a mixture of alpha, beta, delta, and gamma forms. The prolamines found in wheat and rye are known as gliadins. The zeins are extracted from maize gluten. Solutions of zein can be formed into odorless, tasteless, clear, hard almost invisible edible films.

In the present examples the zein coating solution was prepared as a 10% by weight solution, however, solutions could be made having zein at levels of from 1 to 95%, more preferably from 1 to 40% and most preferably from 1 to 20% by weight. The higher the concentration of the zein the more difficult it is to spray the zein solution or to use it in typical coating apparatuses. One formulation for a 10% by weight zein solution is given below in table 1. The process comprised combining a first aliquot of ethanol with the ethyl cellulose and mixing until the ethyl cellulose was solubilized. Methyl cellulose can be substituted for the ethyl cellulose and isopropanol could be used in place of the ethanol. Then the emulsifier, Panodan®, and the zein were added to the ethanol and the solution was mixed until the zein was fully dispersed. Other emulsifiers known in the art can be substituted for the Panodan®. Zein is widely available from manufacturers such as Freeman. Finally, the water and the second aliquot of ethanol were added to bring the final solution weight to 360 grams, for this example, thereby providing a 10% by weight solution of zein.

TABLE 1

| Component | grams | % by weight final |
|---|---|---|
| First ethanol aliquot | 250.00 | 69.44 |
| Ethyl cellulose | 7.60 | 2.11 |
| Emulsifier | 0.39 | 0.11 |
| Zein | 36.00 | 10.00 |
| Water | 44.40 | 12.33 |
| Second ethanol aliquot | 21.61 | 6.01 |

In a first example of using the zein coating solution freeze dried fruit pieces were placed in a fluidized bed system and then sprayed with an algal marine oil containing DHA and EPA to a level of 6% by weight of algal oil. Then half of the samples were coated with the 10% zein solution to a final addition of 20% by weight zein coating. The other half of the samples were not coated with the zein solution. Both sets of samples were then dried in the fluidized bed system and then tested for shelf stability. Shelf stability in the present application and claims is defined as the ability of the food product containing DHA and EPA to be stored without developing a fishy aroma or taste. The samples that were not coated with the zein solution developed a fishy taste and aroma either immediately or within several days making them unpalatable. Samples that had been coated with the zein solution have been found to be stable for over 7 months at 70 to 80° F. These samples have no fishy aroma and taste good even after this long period of storage. This is a dramatic improvement in storage stability. It is anticipated that the zein coating could be reduced to 10% a or less and still provide extended storage stability. Preferably, the zein coating is from 1 to 50% by weight, more preferably from 1 to 40%, and most preferably from 1 to 20% by weight. This process has wide applicability to many food forms including chips, fruit pieces, fruit leathers, crackers, cookies, toaster pastries, ready to eat cereal, baked goods, granola bars, trail mixes, confections, and snack foods. The only limits to use of the fluidized bed are on foods that would be damaged by the action of the fluidizing bed. For such foods the oil containing the DHA and EPA can be applied to the food and then the food can be coated with the zein solution to a level of 10% or more. The DHA and EPA oil could be sprayed on, or a panning process can be used, or a rotating enrobing drum can be used to apply the oil. Then the zein solution can be applied by any of the same methods. The fruit pieces could be freeze dried, dehydrated, pureed or otherwise processed prior to application of the DHA EPA oil. The source of the DHA and EPA could be either the oil form or the powder form suspended in an oil.

In a second example a ready to eat cereal, Smart Start®, was sprayed with an oil. Then a combination of cinnamon seasoning and a powdered algal oil containing DHA and EPA was sprinkled onto the cereal with tumbling to ensure coverage. The final cereal had 7% by weight added oil, 5% by weight added cinnamon seasoning, and 1.04% by weight powdered algal oil to deliver approximately 32 milligrams of DHA per 30 grams of cereal. In one set of samples the powdered algal oil was used as delivered by the manufacturer. In another set of samples the powdered algal oil was initially coated to a final level of 10% by weight zein using the solution described above in table 1 and then dried to remove the ethanol and water. The cereal coated with the powdered algal oil that had not been coated with the zein solution failed almost immediately. It quickly developed a fishy aroma and taste making it unpalatable. The powdered algal oil pre-coated with 10% by weight zein, by way of contrast, was stable on the cereal for over 20 weeks after storage at 85 to 110° F. Even after these elevated temperature storage conditions the cereal had a clean taste with no fishy aroma or taste. Thus, pre-coating the powdered algal oil with zein produced a stable powder containing DHA and EPA. This has wide application to foods and food ingredients. The zein coated powder can be incorporated into many food manufacturing processes with ease. As described above, the zein coated powder can be adhered to foods using an edible oil. Alternatively, the zein coated powder can be added to food doughs during manufacturing to provide a food containing DHA and EPA that is storage stable. These foods could comprise ready to eat cereals, fruit pieces, fruit leathers, chips, snack products, granola bars, trail mix, crackers, cookies, baked goods, toaster pastries and other foods.

The powdered algal oil as obtained from manufacturers is generally quite fine, in the 100 to 400 micron size range, for certain desirable food applications this size range is not ideal. In addition, it can be desirable to create flavored powdered algal oil containing DHA and EPA that is stable. Therefore the inventors also explored ways to create larger agglomerates, flavored agglomerates, and/or seasoned agglomerates containing DHA and EPA that were stable to storage. The uses of such agglomerates are numerous. If they are sized correctly they can be used as additives to ready to eat cereals, trail mixes, chips, granola bars, toaster pastries, baked goods, cookies, crackers, fruit pieces and fruit leathers. In addition, they could be used to provide a seasoning or flavor boost along with the DHA and EPA to a similar range of foods. In the basic process either powdered oil containing DHA and EPA or the oil containing DHA and EPA are added to an agglomerating material in an agglomeration system. The agglomeration parameters are adjusted to produce agglomerates that are suitable and that contain DHA and EPA. If desired, these agglomerates can be sized using sieves and other known in the art sizing procedures. The agglomerates are then coated with the zein solution to the desired level of from 1 to 50%, more preferably from 1 to 40% and most preferably from 1 to 20% by weight zein. These agglomerates containing DHA and EPA are storage stable unlike agglomerates containing DHA and EPA that are not coated with zein. The agglomeration process can also be used to incorporate into the agglomerates powdered DHA and EPA containing oil wherein the powder has previously been coated with zein as described above in the second example. This process eliminates the need to coat the final agglomerate with zein and can be advantageous in certain food applications.

Four classes of base materials were selected for agglomeration testing, these comprised raw flours, pre-gelatinized flours, raw starches, and pre-gelatinized starches. In initial testing it was found that of the four classes only pre-gelatinized flours were able to produce acceptable agglomeration characteristics for the desired product. In general the starches tested were either too powdery to agglomerate or they adsorbed too much water to form low moisture agglomerates. The raw flours were not able to agglomerate. The pre-gelatinized flours that were tested included those from maize, wheat, rice and potatoes. Other pre-gelatinized flours are also expected to work well in the invention. In a first step the pre-gelatinized flour, having a moisture of approximately 25%, was placed into a rotating plastic drum and DHA and EPA containing oil was sprayed onto the pre-gelatinized flour until the desired load had been achieved. Obviously, the DHA and EPA could have been added at this stage as the free flowing powdered form. Any desired seasonings or flavor components are also added at this time. These seasonings can range from sweet seasonings to savory or herb seasonings. Any flavors are acceptable. Once a uniform mixture is achieved whey powder was added at a level of 6% by weight to aid in the agglomeration process. Other agglomeration aids that have been used are modified starches, tapioca starches, modified wheat starches, dextrins and other known agglomeration aids. Preferably the agglomerating aids are use at levels of from 1 to 10% by weight. The uniform mixture of agglomeration base, DHA and EPA source, and agglomeration aid was then weighed and placed into an agglomeration dish. The agglomeration dish is started and the mixture is sprayed with water using a positive displacement pump with a jet nozzle at a rate of 0.29 liters per minute. Dry uniform mixture was added to the agglomerating dish periodically to obtain a dry mix to water ratio of approximately 4.4:1. This process of water and dry addition was continued until all of the dry mixture had been added. The agglomerating dish allows the agglomerated product to fall out of the dish as the agglomeration particle size increases and as its moisture increases. The exiting larger agglomerates were collected as they fell out of the agglomerating dish. The Product exiting the dish had a moisture of approximately 25% and the size of the agglomerates varied. Agglomerated product was dried to a target moisture of approximately 1 to 20%, more preferably from 1 to 15%, and most preferably from 1 to 10% using a fluidized bed dryer. Dried agglomerate was then sieved into desired fractions. A 13% by weight zein coating solution was prepared by combining 112.5 grams of zein with 750 grams of 91% isopropyl alcohol. The agglomerated product was coated with the zein solution to a final level of 13% zein in a tumbler enrober. Once enrobed the product was dried until all the alcohol had been evaporated. The coated agglomerated products had a clean taste with no fishy aroma or taste. Agglomerated product could readily be created in the size range of from about 4000 microns to 1000 microns. Such a size range can be use as a direct add into ready to eat cereals, bars, or dough formulations. The agglomerated product is storage stable for over 20 weeks at ambient temperatures with no development of a fishy taste or aroma. These agglomerates can be used in a wide variety of food forms depending on the size of the agglomerates. As larger agglomerates the additive can be made visible or they can be reduced in size to appear as a seasoning on the food product. As described above any variety of seasonings can be added to the agglomerate to create the desired taste.

All of the processes described above can be use to add omega-3 fatty acids to foods and to create food additives with elevated levels of omega-3 fatty acids. General guidelines suggest that foods should provide from approximately 20 to 150 mg of DHA or EPA per serving. The flexibility of the described processes allows the amount of DHA or EPA to be easily adjustable over a wide range of food platforms.

The foregoing invention has been described in accordance with the relevant legal standards, thus the description is exemplary rather than limiting in nature. Variations and modifications to the disclosed embodiment may become apparent to those skilled in the art and do come within the scope of the invention. Accordingly, the scope of legal protection afforded this invention can only be determined by studying the following claims.

We claim:

1. A method for stabilizing an omega-3 fatty acid comprising the steps of:
a) plating at least one omega-3 fatty acid unto an edible carrier material;
b) applying a zein coating solution to the edible carrier material plated with at least one omega-3 fatty acid; and
d) drying the zein coated carrier material.

2. The method of claim 1 further comprising the step of:
selecting at least one of corn syrup solids, mannitol, calcium carbonate, whey protein isolate, gluten, pre-gelatinized starches, cellulose fiber, carbohydrates, gelatin, flour, grain, bran, and a mixture thereof for the edible carrier material.

3. The method of claim 1 further comprising the step of:
selecting the edible carrier material to have an average particle size of from about 100 to about 400 microns.

4. The method of claim 1 further comprising the step of:
selecting at least one of docosahexaenoic acid, eicosapentaenoic acid, and a mixture thereof as the at least one omega-3 fatty acid.

5. The method of claim 4 further comprising the step of:
sourcing the at least one omega-3 fatty acid from micro algae.

6. The method of claim 4 further comprising the step of:
sourcing the at least one omega-3 fatty acid from a marine animal.

7. The method of claim 1 further comprising the step of:
drying the edible carrier material and the plated at least one omega-3 fatty acid prior to said applying step.

8. The method of claim 7 wherein the drying step is further defined as spray drying.

9. The method of claim 1 further comprising the step of:
selecting the zein solution to have from about 1 to about 20% zein by weight.

10. The method of claim 1 further comprising the step of:
selecting the zein solution to have from about 1 to about 95% zein by weight.

11. The method of claim 10 further comprising the step of:
mixing zein, an alcohol, and water to form the zein solution.

12. The method of claim 11 further comprising the step of:
selecting one of ethanol, isopropanol, and a mixture thereof as the alcohol.

13. The method of claim 11 further comprising the step of:
selecting at least one of ethyl cellulose, methyl cellulose, and a mixture thereof as the zein.

14. The method of claim 1 wherein said applying step is further defined as:
applying the zein coating solution by one of pan coating, spray coating, rotating drum coating, and fluidized bed coating.

15. The method of claim 1 wherein said applying step is further defined as:
applying the zein coating solution to a level of from 1 to 50% by weight based on a total weight.

16. The method of claim 1 wherein said applying step is further defined as:
applying the zein coating solution to a level of from 1 to 40% by weight based on a total weight.

17. The method of claim 1 wherein said applying step is further defined as:
applying the zein coating solution to a level of from 1 to 20% by weight based on a total weight.

18. The method of claim 1 wherein said drying step is further defined as:
drying the zein coated carrier material by one of an air stream, a heated air stream, in a fluidized bed, in an oven, and in an evaporator.

19. The method of claim 1 further comprising the step of:
covering a food product with the dried zein coated edible carrier material.

20. The method of claim 19 wherein said covering step is further defined as:
covering at least one of a ready to eat cereal, a trail mix, a chip, a granola bar, a toaster pastry, a baked good, a cookie, a cracker, a fruit piece, and a fruit leather with the zein coated edible carrier material.

21. The method of claim 19 further comprising the step of:
incorporating the zein coated edible carrier material in one of an oil, an icing solution, and a sugar solution covering the food material.

22. The method of claim 1 further comprising the step of:
incorporating the dried zein coated edible carrier material in a food product.

23. The method of claim 22 wherein said incorporating step is further defined as:
incorporating the zein coated carrier material in one of a ready to eat cereal, a trail mix, a chip, a granola bar, a toaster pastry, a baked good, a cookie, a cracker, a fruit piece, and a fruit leather.

24. A method for creating an agglomerate containing a stabilized omega-3 fatty acid comprising the steps of:
a) providing a pre-gelatinized flour;
b) mixing the pre-gelatinized flour with at least one omega-3 fatty acid and adding it to a agglomerating system;
c) adding water to the mixture of pre-gelatinized flour and at least one omega-3 fatty acid to form a plurality of agglomerates;
d) providing a zein coating solution and applying the zein coating solution to the plurality of agglomerates; and
e) drying the plurality of zein coated agglomerates.

25. The method as recited in claim 24 wherein step a) comprises providing a corn flour, a wheat flour, a rice flour, a potato flour, or a mixture thereof as the pre-gelatinized flour.

26. The method as recited in claim 24 wherein step b) comprises mixing one of an oil containing the at least one omega-3 fatty acid or a free flowing powder containing the at least one omega-3 fatty acid with the pre-gelatinized flour.

27. The method as recited in claim 24 wherein step b) comprises mixing the pre-gelatinized flour with docosahexaenoic acid, eicosapentaenoic acid, or a mixture thereof as the at least one omega-3 fatty acid

28. The method as recited in claim 24 wherein step b) further comprises adding at least one seasoning, or at least one flavoring, or a mixture thereof to the pre-gelatinized flour.

29. The method as recited in claim 24 wherein step b) further comprises adding an agglomerating aid to the pre-gelatinized flour.

30. The method as recited in claim 29 comprising providing at least one of whey powder, modified starch, tapioca starch, modified wheat starch, dextrin, or a mixture thereof as the agglomerating aid.

31. The method as recited in claim 29 comprising providing the agglomerating aid at a level of from 1 to 10% by weight.

32. The method as recited in claim 24 further comprising between steps c) and d) the further step of drying the plurality of agglomerates to a moisture level of from 1 to 20% by weight.

33. The method as recited in claim 24 further comprising between steps c) and d) the further step of drying the plurality of agglomerates to a moisture level of from 1 to 15% by weight.

34. The method as recited in claim 24 further comprising between steps c) and d) the further step of drying the plurality of agglomerates to a moisture level of from 1 to 10% by weight.

35. The method as recited in claim 24 wherein step d) comprises providing a zein coating solution having a level of from 1 to 95% by weight zein.

36. The method as recited in claim 35 wherein step d) comprises providing a zein coating solution having a level of from 1 to 20% by weight zein.

37. The method as recited in claim 24 comprising providing the zein solution as a mixture of zein, an alcohol, and water.

38. The method as recited in a claim 37 comprising providing ethanol, isopropanol, or a mixture thereof as the alcohol.

39. The method as recited in claim 37 comprising providing a zein solution comprising ethyl cellulose, methyl cellulose, or a mixture thereof.

40. The method as recited in claim 24 wherein step d) comprises applying the zein coating solution by one of pan coating, spray coating, rotating drum coating, or fluidized bed coating.

41. The method as recited in claim 24 wherein step d) comprises applying the zein coating solution to a level of from 1 to 50% by weight based on the total weight.

42. The method as recited in claim 24 wherein step d) comprises applying the zein coating solution to a level of from 1 to 40% by weight based on the total weight.

43. The method as recited in claim 24 wherein step d) comprises applying the zein coating solution to a level of from 1 to 20% by weight based on the total weight.

44. The method as recited in claim 24 wherein step e) comprises drying the zein coated carrier material by one of an air stream, a heated air stream, in a fluidized bed, in an oven, or in an evaporator.

45. The method as recited in claim 24 comprising the further step of adding the dried zein coated agglomerate onto a food product.

46. The method as recited in claim 45 comprising adding the zein coated agglomerate onto one of a ready to eat cereal, a trail mix, a chip, a granola bar, a toaster pastry, a baked good, a cookie, a cracker, a fruit piece, or a fruit leather.

47. The method as recited in claim 45 further comprising adhering the zein coated agglomerate onto the food product using one of an oil, an icing solution, or a sugar solution.

48. The method as recited in claim 24 comprising the further step of incorporating the dried zein coated agglomerated into a food product.

49. The method as recited in claim 48 comprising incorporating the zein coated carrier material into one of a ready to eat cereal, a trail mix, a chip, a granola bar, a toaster pastry, a baked good, a cookie, a cracker, a fruit piece, or a fruit leather.

50. The method as recited in claim 24 comprising the further step of sizing the agglomerates either before step d) or after step e).

51. The method as recited in claim 50 wherein the agglomerates are sized to a range of from 2000 to 4000 microns.

52. A method for stabilizing a food product comprising an omega-3 fatty acid comprising the steps of:
a) providing a food product;
b) applying at least one omega-3 fatty acid onto the food product; and
c) providing a zein coating solution and applying the zein coating solution to the food product having the omega-3 fatty acid thereon.

53. The method as recited in claim 52 wherein step a) comprises providing one of a ready to eat cereal, a trail mix, a chip, a granola bar, a toaster pastry, a baked good, a cookie, a cracker, a fruit piece, or a fruit leather.

54. The method as recited in claim 52 wherein step b) comprises applying an oil containing at least one omega-3 fatty acid to the food product.

55. The method as recited in claim 52 wherein step b) comprises applying an oil coating to the food product followed by applying an edible carrier material comprising at least one omega-3 fatty acid to the oil coated food product.

56. The method as recited in claim 55 comprising providing an agglomerate comprising at least one omega-3 fatty acid as the edible carrier material.

57. The method as recited in claim 52 wherein step c) comprises providing a zein coating solution having a level of from 1 to 95% by weight zein.

58. The method as recited in claim 57 wherein step c) comprises providing a zein coating solution having a level of from 1 to 20% by weight zein.

59. The method as recited in claim 52 comprising providing the zein solution as a mixture of zein, an alcohol, and water.

60. The method as recited in a claim 59 comprising providing ethanol, isopropanol, or a mixture thereof as the alcohol.

61. The method as recited in claim 60 comprising providing a zein solution comprising ethyl cellulose, methyl cellulose, or a mixture thereof.

62. The method as recited in claim 52 wherein step c) comprises applying the zein coating solution by one of pan coating, spray coating, rotating drum coating, or fluidized bed coating.

63. The method as recited in claim 52 wherein step c) comprises applying the zein coating solution to a level of from 1 to 50% by weight based on the total weight.

64. The method as recited in claim 52 wherein step c) comprises applying the zein coating solution to a level of from 1 to 40% by weight based on the total weight.

65. The method as recited in claim 52 wherein step c) comprises applying the zein coating solution to a level of from 1 to 20% by weight based on the total weight.

66. The method as recited in claim 52 further comprising after step c) the step of drying the zein coated food product by one of an air stream, a heated air stream, in a fluidized bed, in an oven, or in an evaporator.

67. A stabilized omega-3 fatty acid comprising an edible carrier material, said edible carrier material further comprising at least one omega-3 fatty acid and a zein coating, said zein coating encapsulating said carrier material and said omega-3 fatty acid.

68. The stabilized omega-3 fatty acid as recited in claim 67 wherein said edible carrier material comprises corn syrup solids, mannitol, calcium carbonate, whey protein isolate, gluten, pre-gelatinized starches, cellulose fiber, carbohydrates, gelatin, flour, grain, bran, an agglomerate comprising a pre-gelatinized flour, or mixtures thereof.

69. The stabilized omega-3 fatty acid as recited in claim 68 wherein said pre-gelatinized flour comprises a corn flour, a wheat flour, a rice flour, a potato flour, or a mixture thereof.

70. The stabilized omega-3 fatty acid as recited in claim 67 wherein said at least one omega-3 fatty acid comprises docosahexaenoic acid, eicosapentaenoic acid, or a mixture thereof.

71. The stabilized omega-3 fatty acid as recited in claim 70 wherein said at least one omega-3 fatty acid is sourced from a marine micro algae, a marine animal, or a mixture thereof.

72. The stabilized omega-3 fatty acid as recited in claim 67 wherein said zein coating is present in an amount of from 1 to 50% by weight based on the total weight.

73. The stabilized omega-3 fatty acid as recited in claim 67 wherein said zein coating is present in an amount of from 1 to 40% by weight based on the total weight.

74. The stabilized omega-3 fatty acid as recited in claim 67 wherein said zein coating is present in an amount of from 1 to 20% by weight based on the total weight.

75. The stabilized omega-3 fatty acid as recited in claim 67 wherein said edible carrier material has an average particle size of from 100 to 400 microns.

76. The stabilized omega-3 fatty acid as recited in claim 67 wherein said edible carrier material has an average particle size of from 1000 to 4000 microns.

77. A food product comprising at least one omega-3 fatty acid, said food product encapsulated in a zein coating.

78. The food product as recited in claim 77 wherein said food product comprises a ready to eat cereal, a trail mix, a chip, a granola bar, a toaster pastry, a baked good, a cookie, a cracker, a fruit piece, or a fruit leather.

79. The food product as recited in claim 77 wherein said at least one omega-3 fatty acid comprises docosahexaenoic acid, eicosapentaenoic acid, or a mixture thereof.

80. The food product as recited in claim 77 wherein said at least one omega-3 fatty acid is sourced from a marine micro algae, a marine animal, or a mixture thereof.

81. The food product as recited in claim 77 wherein said at least one omega-3 fatty acid is in the form of an oil coating on the food product.

82. The food product as recited in claim 77 wherein said at least one omega-3 fatty acid is in the form of an edible carrier material comprising an omega-3 fatty acid on the food product.

83. The food product as recited in claim 77 wherein said zein coating is present in an amount of from 1 to 50% by weight based on the total weight.

84. The food product as recited in claim 77 wherein said zein coating is present in an amount of from 1 to 40% by weight based on the total weight.

85. The food product as recited in claim 77 wherein said zein coating is present in an amount of from 1 to 20% by weight based on the total weight.

86. A food product comprising an edible carrier material comprising at least one omega-3 fatty acid, said edible carrier material encapsulated in a zein coating.

87. The food product recited in claim 86 wherein said food product comprises a ready to eat cereal, a trail mix, a chip, a granola bar, a toaster pastry, a baked good, a cookie, a cracker, a fruit piece, or a fruit leather.

88. The food product as recited in claim 86 wherein said at least one omega-3 fatty acid comprises docosahexaenoic acid, eicosapentaenoic acid, or a mixture thereof.

89. The food product as recited in claim 86 wherein said at least one omega-3 fatty acid is sourced from a marine micro algae, a marine animal, or a mixture thereof.

90. The food product as recited in claim 86 wherein said edible carrier material comprises corn syrup solids, mannitol, calcium carbonate, whey protein isolate, gluten, pre-gelatinized starches, cellulose fiber, carbohydrates, gelatin, flour, grain, bran, agglomerated pre-gelatinized flour or mixtures thereof.

91. The food product as recited in claim 86 wherein said edible carrier material has an average particle size of from 100 to 400 microns.

92. The food product as recited in claim 86 wherein said edible carrier material has an average particle size of from 1000 to 4000 microns.

93. The food product as recited in claim 86 wherein said edible carrier material is adhered to an outside of said food product.

94. The food product as recited in claim 93 wherein said edible carrier material is adhered to said outside of said food product by an oil coating, an icing, or a sugar syrup solution.

95. The food product as recited in claim 86 wherein said edible carrier material is incorporated into said food product.

96. The food product as recited in claim 86 wherein said zein coating is present in an amount of from 1 to 50% by weight based on the total weight.

97. The food product as recited in claim 86 wherein said zein coating is present in an amount of from 1 to 40% by weight based on the total weight.

98. The food product as recited in claim 86 wherein said zein coating is present in an amount of from 1 to 20% by weight based on the total weight.

99. The food product as recited in claim 86 wherein said edible carrier material further comprises a flavor, a seasoning, or a mixture thereof.
